SEQUENCE LISTING (10 sequences)

SEQ ID NO: 1 - DNA, 21 bp, Artificial
  RNA molecule containing two dT at 3′-terminus
ccgcaacuacaguaagcugtt

SEQ ID NO: 2 - DNA, 21 bp, Artificial
  RNA molecule containing two dT at 3′-terminus
cagcuuacuguaguugcggtt

SEQ ID NO: 3 - DNA, 21 bp, Artificial
  RNA molecule containing two dT at 3′-terminus
gcgcgcuuuguaggauucgtt

SEQ ID NO: 4 - DNA, 21 bp, Artificial
  RNA molecule containing two dT at 3′-terminus
cgaauccuacaaagcgcgctt

SEQ ID NO: 5 - DNA, 26 bp, Artificial
  sense primer
caccatgcctatgttcatcgtgaaca

SEQ ID NO: 6 - DNA, 29 bp, Artificial
  antisense primer
gcccgggctcaagcgaaggtggaaccgtt

SEQ ID NO: 7 - DNA, 23 bp, Artificial
  sense primer
gcgggaatcctgaagaagtctac

SEQ ID NO: 8 - DNA, 20 bp, Artificial
  antisense primer
ttggtccaggaggaaaagcg

SEQ ID NO: 9 - DNA, 26 bp, Artificial
  sense primer
tgaaggtcggtgtcaacggatttggc

SEQ ID NO: 10 - DNA, 24 bp, Artificial
  antisense primer
catgtaggccatgaggtccaccac

(19) United States
(12) Patent Application Publication
(10) Pub. No.: US 2008/0070857 A1
(43) Pub. Date: Mar. 20, 2008

(54) Pharmaceutical Agents for Preventing Metastasis of Cancer

(76) Inventors: Jun Nishihira, Hokkaido (JP); Sun Bailong, Hokkaido (JP); Yoshikazu Koyama, Hokkaido (JP); Kazunori Asada, Hokkaido (JP)

Correspondence Address: MORGAN LEWIS & BOCKIUS LLP, 1111 PENNSYLVANIA AVENUE NW, WASHINGTON, DC, 20004, US

(21) Appl. No.: 11/884,210

(22) PCT Filed: Feb. 14, 2006
(86) PCT No.: PCT/JP06/02920

§ 371 Date: Aug. 13, 2007

Related U.S. Application Data
(60) Provisional application No. 60/653,246, filed on Feb. 14, 2005.

(51) Int. Cl. A61K 31/711; A61P 35/04; C07H 21/04

(52) U.S. Cl. 514/44; 536/24.5

(57) ABSTRACT

This invention relates to a pharmaceutical agent for preventing metastasis of cancer, which comprises a substance that inhibits expression of macrophage migration inhibitory factor (MIF). Such substance includes siRNA of MIF.

### TECHNICAL FIELD

This invention relates to a pharmaceutical agent for preventing metastasis of cancer, which comprises a substance that inhibits expression of macrophage migration inhibitory factor (MIF).

### BACKGROUND ART

Macrophage migration inhibitory factor (MIF) was originally identified as a T-cell-derived lymphokine (Proc. Natl. Acad. Sci. U.S.A. 1966; 56: 72-7; Science 1966; 153: 80-2). Recent studies have revealed that MIF is ubiquitously expressed in various types of cells, and has been reevaluated as a pluripotent cytokine involved in a broad-spectrum immune system (FASEB. J. 1996; 7: 19-24; J. Interferon. Cytokine Res 2000; 20: 751-62). In brief, MIF has been recognized as a pituitary hormone released in response to an array of stimuli (Nature 1993; 365: 756-9), a proinflammatory cytokine released mainly by macrophages (J. Exp Med 1994; 179: 1895-902), and a T-cell activator essential for immune responses (Proc Natl Acad Sci U.S.A. 1996; 93: 7849-54). MIF is a unique protein induced by glucocorticoids and counteracting their anti-inflammatory and immunosuppressive functions (Nature 1995; 377: 68-71). On the other hand, Meyer-Siegler et al. reported that MIF mRNA levels in prostatic adenocarcinoma and its metastatic tumors were higher than those in normal prostatic tissues (Urology 1996; 48: 448-52). Moreover, MIF is known to be involved in angiogenesis, tumor growth and metastasis (Int J Mol Med 2003; 12: 633-41). These observations prompted us to investigate the involvement of MIF in tumor invasion and metastasis.

The liver is a common site of metastasis for a number of tumor types, especially colon cancer. Colorectal liver metastasis is associated with a very poor prognosis; most patients die within two years of diagnosis despite the availability of numerous therapies. In order to improve the choice of therapeutic strategy, it is critical that the mechanism of invasion and metastasis of cancer cells be clarified. Cell migration and invasion are regulated by a combination of different processes, including the contraction of actomyosin, the formation of stress fibers, and the turnover of focal adhesions with Rho activity (Science 1999; 286: 1102-3; J Cell Biol 1996; 133: 1403-15). Lysophosphatidic acid (LPA) may play an important role in the development or progression of colon cancer (Cancer Res 2003; 63: 1706-11), and induce many cellular effects, including mitogenesis, secretion of proteolytic enzymes (FASEB J 1998; 12: 1589-98). In the processes of tumor invasion and metastasis, migration activity is accompanied by stress fiber formation and focal adhesion assembly through an Rho/Rho-associated kinase pathway (Gynecol Oncol 2002; 87: 252-9).

Under these situations, there is a need of developing an effective pharmaceutical agent for treating cancer or preventing metastasis of cancer.

### DISCLOSURE OF INVENTION

### SUMMARY OF THE INVENTION

In view of the above, the inventors analyzed the cellular effects of MIF siRNA on tumor invasion and metastasis of MIF in tumor invasion and metastasis, and completed the invention. The present invention provides:

(1) A pharmaceutical agent for preventing metastasis of cancer, which comprises a substance that inhibits expression of MIF;

(2) A pharmaceutical agent according to (1) above, wherein the substance is selected from siRNA of MIF, antisence of MIF, ribozyme of MIF and a compound that inhibits expression of MIF;

(3) A pharmaceutical agent according to (1) above, wherein the substance is siRNA of MIF;

(4) A pharmaceutical agent according to any one of (1) through (3) above, wherein the cancer is prostatic adenocarcinoma or colon cancer;

(5) A method of preventing metastasis of cancer, which is characterized by using a substance that inhibits expression of MIF;

(6) A method according to (5) above, wherein the substance is selected from siRNA of MIF, antisence of MIF, ribozyme of MIF and a compound that inhibits expression of MIF;

(7) A method according to (5) above, wherein the substance is siRNA of MIF;

(8) A method according to any one of (5) through (7) above, wherein the cancer is prostatic adenocarcinoma or colon cancer;

(9) Use of a substance that inhibits expression of MIF for manufacturing a pharmaceutical agent for preventing metastasis of cancer;

(10) Use according to (9) above, wherein the substance is selected from siRNA of MIF, antisence of MIF, ribozyme of MIF and a compound that inhibits expression of MIF;

(11) Use according to (9) above, wherein the substance is siRNA of MIF; and

(12) Use according to any one of (9) through (11) above above, wherein the cancer is prostatic adenocarcinoma or colon cancer.

### BRIEF DESCRIPTION OF DRAWINGS

FIG. 1 shows the results of dose-response study of MIF siRNA and LPA on MIF expression: (A) Western blot analysis; and (B) Northern blot analysis.

FIG. 2 shows the results of effect of MIF siRNA on MIF production.

FIG. 3 shows effect of MIF siRNA on LPA-induced cell invasion.

FIG. 4 shows the results of in vivo metastasis assay: (A) Macroscopic images of metastatic nodules in the livers; (B) Quantitative analysis of the number of liver metastatic nodules; (C) Quantitative analysis of the liver weights; and (D) Western blot analysis using whole livers to assess MIF protein levels in livers with or without metastatic tumors.

FIG. 5 shows histological changes in the liver surrounding metastatic foci, which were examined to evaluate the effectiveness of MIF siRNA by H-E staining.

FIG. 6 shows the results of immunohistochemical analysis for angiogenesis.

FIG. 7 shows effect of MIF siRNA on activation of Rho.

FIG. 8 shows effect of MIF siRNA on LPA-stimulated tyrosine-phosphorylation of FAK: (A) The amount of phosphorylated FAK by immunoblot analysis; and (B) The effect of MIF siRNA on FAK phosphorylation in the absence of LPA.

FIG. 9 shows effect of siRNA on integrin β1 production induced by LPA.

FIG. 10 shows effect of MIF siRNA on the production of MMP-13 induced by LPA.

### BEST MODE FOR CARRYING OUT OF THE INVENTION

Macrophage migration inhibitory factor (MIF) plays an important role not only in the immune system, but also in tumorigenesis. The inventors investigated the potential role of MIF in association with tumor invasion and metastasis.

To assess the function of MIF, the inventors knocked down the MIF mRNA using small interfering RNA (siRNA). Twenty-one base siRNA specific for the mRNA sequence of mouse MIF was introduced to a murine colon cancer cell line, colon 26. Tumor cell invasion was evaluated using a transwell method (8-μM pores) coated with Matrigel on the upperside membrane and with fibronectin (FN) on the underside membrane. Moreover, the inventors investigated the signal transduction of LPA relevant to the Rho-dependent pathway, and further examined the effect of MIF siRNA on this signal transduction system. In vivo, the tumor cells were pretreated with MIF siRNA and injected into the portal vein, and the effects on metastasis to the liver were evaluated.

The inventors found that MIF siRNA markedly reduced the invasion of the cells from the upperside and lowerside membranes. The inventors revealed that the Rho-dependent pathway activated by lysophosphatidic acid (LPA) was suppressed by MIF siRNA. Next, the inventors found that the tyrosine-phosphorylation of focal adhesion kinase, and LPA-induced expressions of integrin β1 were significantly suppressed by MIF siRNA. In vivo, metastasis to the liver was significantly inhibited by pretreatment of the cells with MIF siRNA.

Taken together, these results indicate that MIF promotes tumor invasion and metastasis via the Rho-dependent pathway. The present invention is based on these findings.

This invention provides a pharmaceutical agent for preventing metastasis of cancer, which comprises a substance that inhibits expression of macrophage migration inhibitory factor (MIF). Hereinafter, it is described in detail.

The term “metastasis of cancer” means that the cancer spreads to the other organs, finally to the whole body as cancer progresses. For metastasis of cancer, two forms: disseminated and hematogenous metastasis is known in persons skilled in the art. The disseminated metastasis indicates that the cancer cell invades surrounding tissues and expands into the abdominal cavity through the body fluid, and the hematogenous-metastasis means that the cancer cell permeates surrounding vein and lymphatic and spreads to the distant organ through the blood and the lymph fluid. MIF is one of cytokines, which participates in the invasion and the metastasis of cancer cells.

Throughout the specification, the term “MIF gene” means a gene that is registered as the Accession No. S73424 on the NCBI Nucleotide Database and consists of 348 nucleotides. However, for example, a variant is included, wherein one or more nucleotides are substituted, deleted, added or inserted on the nucleotide sequence of the gene.

Herein, the term “inhibition of gene expression (or merely ‘expression’)” means the fact that production of a protein encoded by the gene is inhibited by inhibiting any process of events from gene through protein including transcription and translation.

In general, to inhibit gene expression, (i) antisense oligonucleotides, (ii) ribozymes, (iii) siRNA, (iv) other substances that inhibit expression or the like can be used. Preferably, in the present invention, siRNA can be used.

Antisense Oligonucleotides

Antisense oligonucleotides are short single-strand molecules that are complementary to the target mRNA and typically have 10-50 mers in length, preferably 15-30 mers in length, more preferably 18-20 mers in length. Antisense oligonucleotides are preferably designed to target the initiator codons, the transcriptional start site of the targeted gene or the intron-exon junctions.

Antisense oligonucleotides are thought to inhibit gene expression through various mechanisms: (1) Degradation of the complexes between target RNA/DNA oligonucleotide by RNase H. The latter is a ubiquitous nuclear enzyme required for DNA synthesis, which functions as an endonuclease that recognizes and cleaves the RNA in the duplex. Most types of oligonucleotides, but not all, from complexes with mRNA that direct the cleavage by RNase H; (2) Inhibition of translation by the ribosomal complexes; (3) Competition for mRNA splicing when oligonucleotides are designed against intron-exon junctions.

Ribozymes

Ribozymes are single stranded RNA molecules retaining catalytic activities. Their structures are based on naturally occurring site-specific, self-cleaving RNA molecules. Five classes of ribozymes have been described based on their unique characters, i.e. the Tetrahymena group I intron, RNase P, the hammerhead ribozyme, the hairpin ribozyme and the hepatitis delta virus ribozyme.

The hammerhead ribozyme at about 40 nucleotides shares similarities with the shape of a hammerhead. They are the most common and the smallest of the naturally occurring ribozymes. Their normal function is to process multimeric viral RNAs into monomers. Hammerhead motif was shown as the most efficient self-cleaving sequence that can be isolated from randomized pools of RNA. Hammerhead ribozymes-like oligonucleotides are presently used because of their effectiveness and their short sequence.

The hammerhead ribozyme consists of three short stems: stems I and II are designed to anneal with a specific RNA sequence, whereas stem-loop III contains a catalytic cleavage site. Once bound, hammerhead ribozyme cleaves the phosphodiester backbone of the target RNA by a transesterification reaction. The catalytic moiety of ribozymes recognize specific 5′-UH-3′ sites of the mRNA target, where U is a uracile and H is an adenine, cytosine or uracile. The mechanism of cleavage is free of any protein factor, but requires the presence of bivalents metal cations, e.g. Mg²⁺. Once they have cleaved their target, ribozymes are released from their mRNA target and are free to cleave another mRNA molecule.

It has been shown that unmodified and modified (2′-O-methyl and phosphorothioate) synthetic ribozymes can be used to inhibit specifically gene expression in vitro. It has also been shown that inhibition of gene expression can be also achieved by administration of synthetic modified ribozymes to living organisms.

siRNA

RNA interference (RNAi), quelling or post-transcriptional gene silencing (PTGS) designate a phenomenon by which dsRNA specifically suppresses expression of a target gene at post-translational level. This mechanism, which was originally discovered in Caenorhabditis elegans, has now been found in a large number of organisms including fungi, parasites, Planaria, Hydra, Drosophila, zebra fish, plants, and mammalians.

In normal conditions, RNAi is initiated by double-stranded RNA molecules (dsRNA) of several thousands of base pair length. In vivo, dsRNA introduced into a cell is cleaved into a mixture of short dsRNA molecules called short interfering RNA (siRNA). The enzyme that catalyzes the cleavage, Dicer, is an endo-RNase that contains RNase III domains. The siRNAs produced by Dicer are 21-23 bp in length in mammalian cells.

Acquisition of the siRNA Sequence of the Present Invention

For an effective selection of siRNAs on the target gene, the inventors can utilize a regression analysis using learning algorithm developed by Dr. Mitsuhiro Tada of Hokkaido University.

A Pharmaceutical Agent for Preventing Metastasis of Cancer

When a substance that inhibits expression of MIF (an active ingredient such as siRNA) is used as the pharmaceutical agents for preventing metastasis of cancer, a conventional means may be applied to making pharmaceutical preparations. For example, the afore-mentioned substance may be prepared into tablets, capsules, elixirs, microcapsules, sterile solutions, suspensions, etc.

Since the thus obtained preparation is safe and low toxic, it can be administered orally or parenterally to human or warm-blooded animal (e.g., mice, rats, rabbits, sheep, swine, bovine, horses, chickens, cats, dogs, monkeys, etc.).

The dose of the active ingredient varies depending on activity, target disease, subject to be administered, method for administration, etc.; for example, when the active ingredient of the present invention is orally administered, the dose is normally about 0.1 to about 100 mg, preferably about 1.0 to about 50 mg, more preferably about 1.0 to about 20 mg per day for adult (as 60 kg body weight). In parenteral administration, the single dose varies depending on subject to be administered, target disease, etc., it is advantageous to administer the active ingredient of the present invention intravenously at a daily dose of about 0.01 to about 30 mg, preferably about 0.1 to about 20 mg, more preferably about 0.1 to about 10 mg for adult (as 60 kg body weight). For other animal species, the corresponding dose as converted per 60 kg weight can be administered.

### EXAMPLES

### Experimental Example 1

### Materials

All of the following materials were obtained from commercial sources. Nitrocellulose membrane filters were purchased from Millipore (Bedford, Mass.); the ECL Western blotting detection system from Amersham Biosciences (Piscataway, N.J.); horseradish peroxidase (HRP)-conjugated goat anti-rabbit IgG antibody and Micro BCA protein assay kit from Pierce (Rockford, Ill.); Konica HRP-1000 immunostaining kits from Konica (Tokyo, Japan); Diff Quick solution from International Reagent Corp. (Kobe, Japan); E. coli BL21/DE3 from Novagen (Madison, Wis.); fetal calf serum (FCS) and RPMI-1640 from Gibco BRL (Grand Island, N.Y.); the Rho pull-down assay kit from Cytoskeleton (Denver, Colo.); Protein A Sepharose from Pharmacia (Uppsala, Sweden); Effectene transfection reagent from Qiagen (Valencia, Calif.); and 1-oleoyl-2-lysophosphatidic acid (LPA) and type I collagen from Sigma (St. Louis, Mo.). All other chemicals used were of analytical grade. Recombinant mouse MIF was expressed in E. coli BL21/DE3 and purified as described previously (Biochim Biophys. Acta 1995; 1247: 159-62). A polyclonal anti-mouse MIF antibody was generated by immunizing New Zealand White rabbits with recombinant mouse MIF. The IgG fraction was prepared using Protein A Sepharose according to the manufacturer's protocol.

### Experimental Example 2

### Mice

Four-week-old female BALB/c mice were purchased from Clea (Tokyo, Japan) and acclimatized for at least 1 week. They were used at 6-8 weeks of age. Mice were maintained under a 12 h light/dark cycle (lights on from 6:00 AM to 6:00 PM) at a temperature of 20-22° C. Food and water were available ad libitum. Animal studies conformed to the Regulations for Animal Experiments of the Institute for Animal Experimentation, Hokkaido University Graduate School of Medicine.

### Experimental Example 3

### Cell Culture

The colon 26 cell line, established from BALB/c mice, was a generous gift from Dr. T. Kataoka (Cancer Chemotherapy Center, Tokyo, Japan). Colon 26 cells were cultured in RPMI-1640 medium containing 10% heat-inactivated FCS at 37° C. under 5% CO₂, and subcultured every 3 days. For all experiments, logarithmically growing cells were used.

### Experimental Example 4

### Transfection with siRNA

The RNAi technique is used for down-regulating the expression of a specific gene in living cells by introducing a homologous double-stranded RNA, and 21 base siRNAs are potent mediators of the RNAi effect in mammalian cells (16). The nucleotide sequences of dsRNA and complimentary dsRNA for mouse mRNA were 5′-CCGCAACUACAGUAAGCUGdTdT-3′ (SEQ ID NO: 1) and 5′-CAGCUUACUGUAGUUGCGGdTdT-3′ (SEQ ID NO: 2), respectively. As a control RNA duplex (scramble RNA), 5′-GCGCGCUUUGUAGGAUUCGdTdT-3′ (SEQ ID NO: 3) and 5′-CGAAUCCUACAAAGCGCGCdTdT-3′ (SEQ ID NO: 4) were used. Colon 26 cells (2×10⁵ cells) in culture dishes (60 mm in diameter) containing RPMI-1640 with FCS (10%) were transfected with either the MIF siRNA or the control RNA duplex using Effectene according to the manufacturer's protocol. After 48 h, the culture medium was removed and the cells were cultured under serum-free condition for 18 h prior to stimulations.

### Experimental Example 5

### RNA Extraction and Semiquantitative RT-PCR

For RNA extraction, the rats were killed after anesthesia with sodium pentobarbital at 4, 7, 14, 21, and 28 days postoperatively, and normal and fractured femora were harvested. The tissues were immediately frozen in liquid nitrogen and stored at −80° C. until use in the RNA isolation. Total RNA was extracted using Trizol (Gibco BRL, Rockville, Md.) according to the manufacturer's protocol. Total RNA (3.0 μg/ml) was incubated at 65° C. for 10 min for denaturation. Denatured RNA (3.0 μg/ml), 5×RT buffer (1×RT buffer: 50 mM Tris, pH 8.3, 50 mM KCl, 8.0 mM MgCl₂, and 10 mM dithiothreitol), 2.5 mM deoxyoligonucleoside triphosphate (dNTP), 100 pM oligo-dT, and 0.5 μl of Monkey murine leukemia virus reverse transcriptase (Gibco BRL), and 0.4 μl RNase inhibitor were incubated at room temperature for 10 min. After this process, 10 μl of this mixture was incubated at 42° C. for 1 h. Three μl of the double-strand product was then mixed with 10×Taq/RT buffer (1×Taq/RT buffer: 10 mM Tris, pH 8.3, 50 mM KCl, 1.5 mM MgCl₂, 0.01% gelatine, and 2.0 mM dithiothreitol), 500 μM dNTP mix, 25 mM MgCl₂, 500 μM of each sense and antisense oligonucleotide, and 0.25 μl Taq polymerase (Promega, Madison, Wis.). The PCR primers for amplification of rat MIF, MMP-13, and glyceroaldehyds-3-phosphate dehydrogenase (GAPDH) were designed as follows: MIF (360 bp), sense primer 5′-CACCATGCCTATGTTCATCGTGAACA-3′ (SEQ ID NO: 5) and antisense primer 5′-GCCCGGGCTCAAGCGAAGGTGGAACCGTT-3′ (SEQ ID NO: 6); MMP-13 (424 bp), sense primer 5′-GCGGGAATCCTGAAGAAGTCTAC-3′ (SEQ ID NO: 7) and antisense primer 5′-TTGGTCCAGGAGGAAAAGCG-3′ (SEQ ID NO: 8); GAPDH (983 bp), sense primer 5′-TGAAGGTCGGTGTCAACGGATTTGGC-3′ (SEQ ID NO: 9) and antisense primer 5′-CATGTAGGCCATGAGGTCCACCAC-3′ (SEQ ID NO: 10). After a heating step at 94° C. for 5 min, PCR was performed for MIF, MMP-13, and GAPDH. Amplification was carried out under the following conditions: MIF, 94° C. for 1 min, 55° C. for 1 min, and 72° C. for 30 s for 30 cycles; MMP-13, 94° C. for 1 min, 58° C. for 2 min, and 72° C. for 2 min for 22 cycles; GAPDH, 94° C. for 1 min, 58° C. for 1 min, and 72° C. for 1 min for 18 cycles. Following these steps, a final extension at 72° C. for 7 min for these three samples using a thermal cycler (Perkin Elmer, Norwalk, Conn.). The products were analyzed after separation by gel electrophoresis (2% agarose) and analyzed by scanning densitometry to produce a standard curve that determined the linear range of quantifiable reaction products. GAPDH mRNA expression was used as a loading control.

### Experimental Example 6

### Histology and Immunohistochemical Analysis

Liver tissues were fixed in 10% PBS-buffered formalin, and paraffin sections were stained with hematoxylin and eosin and examined by light microscopy to assess the histologic changes. Immunohistochemical analysis was performed using a Vectastain ABC kit according to the manufacturer's protocol. Briefly, liver tissues obtained from mice were surgically excised at day 14 after inoculation. The livers were immersed in 10% PBS-buffered formalin. Sections were treated with methanol containing 0.3% hydrogen peroxide for 30 min to inactivate endogenous peroxidase. After washing with PBS, sections were incubated with a blocking solution for 30 min. An anti-mouse monoclonal antibody against CD31 was used as a marker for macrophages. The sections were incubated overnight at 4° C. with the anti-CD31 antibody (1:100 dilution) and the reaction was visualized using 3,3′-diaminobenzidine tetrachloride containing 0.01% hydrogen peroxide. After counterstaining with hematoxylin, sections were microscopically examined and the numbers of positively stained cells were counted. Ten fields per each section were counted using x 100 objectives (n=3).

### Experimental Example 7

### Immunoblot Analysis

Cells (1×10⁶ cells) were disrupted with a Polytron homogenizer (Kinematica, Lucerne, Switzerland). The protein concentrations of the cell homogenates were quantitated using a Micro BCA protein assay reagent kit. Equal amounts of homogenates were dissolved in 20 μl of Tris-HCL, 50 mM (pH 6.8), containing 2-mercaptoethanol (1%), sodium dodecyl sulfate (SDS) (2%), glycerol (20%), and bromphenol blue (0.04%), and heated at 100° C. for 5 min. The samples were subjected to SDS-polyacrylamide gel electrophoresis (SDS-PAGE) and transferred electrophoretically onto a nitrocellulose membrane (Proc Natl Acad Sci U.S.A. 1979; 76: 4350-4; and Anal Biochem 1987; 166: 368-79). The membranes were blocked with 5% nonfat dry milk and 0.1% Tween in phosphate-buffered saline (PBS), probed with anti-FAK (pY397) phosphospecific antibody, and reacted with the anti-rabbit IgG antibody coupled with horseradish peroxidase. To assay for integrin β1, the membrane was probed with the anti-mouse CD29 (integrin β1 chain), and reacted with the rat anti-rat IgG antibody coupled with horseradish peroxidase. For the assay with MMP-13, anti-mouse MMP-13 antibody was used as a primary antibody, and anti-mouse IgG antibody was used as a secondary antibody. The resultant complexes were processed for detection by enhanced chemiluminescence using an ECL Western blotting detection system according to the manufacturer's protocol. For the assay with MIF, the inventors performed immunoblot analysis in a similar manner using the anti-rat MIF polyclonal antibody and the anti-rabbit IgG antibody coupled with horseradish peroxidase as primary and secondary antibodies, respectively. After the reaction, proteins were visualized with a Konica HRP-1000 immunostaining kit as recommended in the manufacturer's protocol.

### Experimental Example 8

### Analysis of Invasion

Invasion of colon 26 cells was measured by invasion of cells through Matrigel-coated transwell inserts (Becton Dickinson, Franklin Lakes, N.J.). In brief, transwell inserts with 8-μM pores were coated on the upperside with Matrigel (40 μg/well) and the underside with fibronectin (10 μg/well) by passive adsorption. After treatment with MIF siRNA or control siRNA, the cells were cultured under serum-free condition for 24 h. Following this, cells (2×10⁵/100 μl/well) were plated in the upper chamber in the presence of LPA and allowed to invade for 24 h. Non-invasive cells were removed from the upper chamber with a cotton swab, and migrating cells adhering to the underside of the filter were fixed, stained with Diff Quick solution, and enumerated using an ocular micrometer; a minimum of 10 random fields/filter were counted. All of the experiments were performed independently and in triplicate.

### Experimental Example 9

### In Vivo Metastasis Assay

After 48 h of treatment with MIF siRNA and control siRNA, the colon 26 cells were harvested with 1 mM EDTA in PBS, washed three times with serum-free RPMI-1640, and resuspended in PBS to a final concentration of 1×10⁴ cells/100 μl. Cells were injected into the portal vein of BALB/c mice that had been anesthetized with ether and laparotomized. The inventors divided mice into three groups: (i) a negative control group in which colon 26 cells were injected into the portal vein; (ii) a positive control group in which colon 26 cells treated with control siRNA were injected into the portal vein; and (iii) a test sample group in which colon 26 cells treated with MIF siRNA were injected into the portal vein. Fourteen days after inoculation with tumor cells, the mice were killed and the number of metastatic colonies in each liver was macroscopically counted. At the same time, the liver weight was recorded to evaluate tumor metastasis.

### Experimental Example 10

### Rho Pull-Down Assay

The Rho pull-down assay was performed according to the manufacturer's protocol. Briefly, cells (5×10⁵/well) were plated, allowed to attach for 12 h, treated with MIF siRNA or control siRNA for 48 h, and cultured under serum-free conditions for 24 h. After incubation, cells were stimulated with 20 μM LPA for 30 min and 60 min, washed twice with PBS, and lysed in immunoprecipitation assay buffer. Cell lysates were clarified by centrifugation, and equal volumes of lysates were incubated with Rhotekin RBD-agarose beads (30 μg) at 4° C. for 45 min. The beads were washed three times with washing buffer. Bound Rho proteins were detected by Western blot analysis using a monoclonal antibody against Rho. Western blot analysis of the total amount of Rho in cell lysates was performed for the comparison of Rho activity (level of GTP-bound Rho) in different samples.

### Experimental Example 11

### Statistical Analysis

All of the statistical analyses were carried out using Student's t-test. Values of p<0.05 were considered to indicate statistical significance.

### Example 1

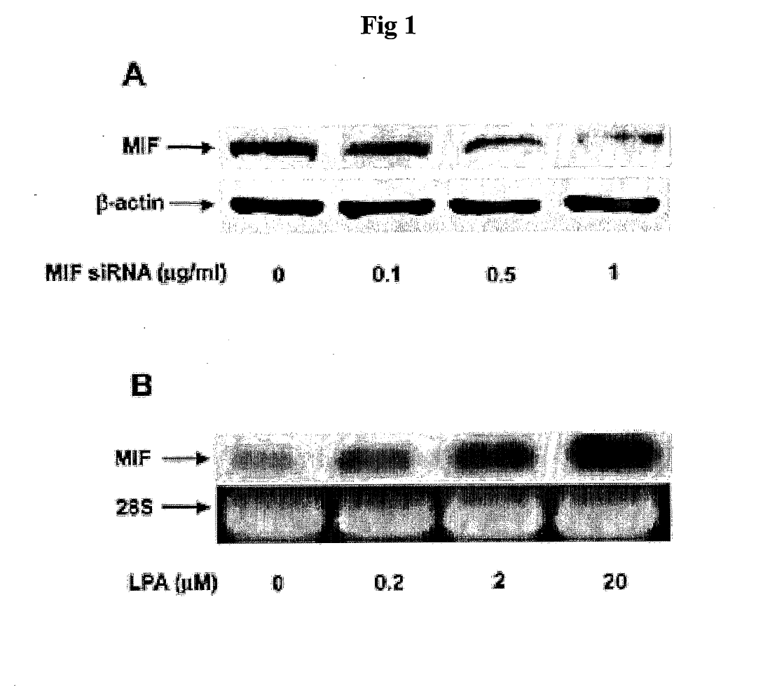

The inventors carried out a dose-dependent study to assess the effects of MIF siRNA on MIF protein expression by Western blot analysis using colon 26 cells. These cells (1×10⁶ cells/5 ml), in FCS-free RPMI-1640 containing LPA (20 μM), were treated with various doses of MIF siRNA, ranging from 0.1 to 1 μg/ml. After 48 h, the cells were washed and subjected to Western blot analysis to measure levels of MIF protein. Specifically, Western blot analysis was carried out to assess MIF siRNA's effect on MIF protein expression. Various doses of MIF siRNA, ranging from 0.1 to 1 μg/ml, were added to colon 26 cells (1×10⁶ cells/5 ml) in FCS-free RPMI-1640 containing LPA (20 μM). After 48 h, the cells were washed and subjected to Western blot analysis to detect MIF protein. Also, Northern blot analysis was performed to examine the expression of MIF mRNA in response to LPA. Various doses of LPA, ranging from 0.2 to 20 μM, were added to colon 26 cells (1×10⁷ cells/10 ml) in FCS-free RPMI-1640. After 24 h, the total RNA was extracted and subjected to Northern blot analysis. A band for 28S ribosomal RNA stained with ethidium bromide is shown at the bottom of each lane. The inventors found that MIF siRNA significantly suppressed MN expression (FIG. 1A). Second, Northern blot analysis was carried out to assess the expression of MIF mRNA in response to LPA. LPA, ranging from 0.2 to 20 μM, was added to colon 26 cells (1×10⁷ cells/10 ml) in FCS-free RPMI-1640. After 24 h culture, MIF mRNA was found to have increased in a dose-dependent manner (FIG. 1B).

### Example 2

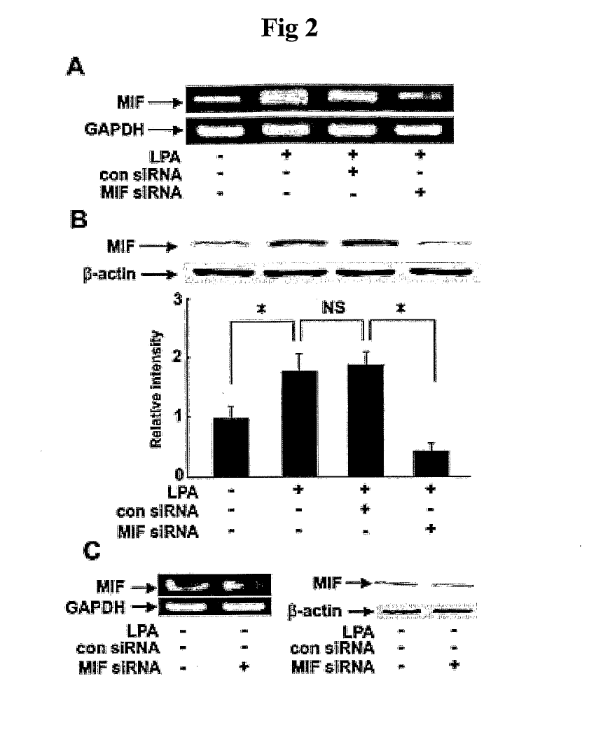

The expression levels of MIF mRNA and MIF protein of colon 26 cells were examined by RT-PCR and Western blot analysis, respectively. Specifically, the inventors examined the effect of LPA (20 μM) on MIF mRNA expression and MIF protein in colon 26 cells, and assessed the effect of MIF siRNA (1 μg/ml). A scramble siRNA (1 μg/ml) was used as controls (con siRNA). As for MIF mRNA expression (A), total RNA was isolated and RT-PCR analysis was performed as described in EXPERIMENTAL EXAMPLE 5. Expression of GAPDH mRNA is presented as controls. As for MIF protein (B), equal amounts of cell lysates (40 μg protein) were applied to each lane, and immunoblot analysis was performed using the anti-MIF antibody. The quantity of MIF normalized by β-actin is shown below each lane. *Values are statistically significant (P<0.05). MIF siRNA's effects on both MIF mRNA expression and MIF protein levels were assessed in the absence of LPA by RT-PCR and Western blot analysis, respectively (C). MF mRNA was upregulated by LPA (20 μM) which was suppressed by the siRNA treatment (1 μg/ml) for 48 h, whereas it was not significantly affected by control siRNA (FIG. 2A). As a control, the expression of GAPDH was measured. The inventors next examined the effect of MIF siRNA on MIF protein production. The MIF content increased by LPA (20 μM) was significantly reduced by approximately 50% by MIF siRNA as assessed by immunoblot analysis (FIG. 2B). The amount of β-actin was measured as a control. In the absence of LPA, MIF siRNA had no apparent effect on MIF mRNA expression (FIG. 2C). Consistent with this result, the inventors found that MIF contents in the culture media were decreased by the MIF siRNA treatment (data not shown).

### Example 3

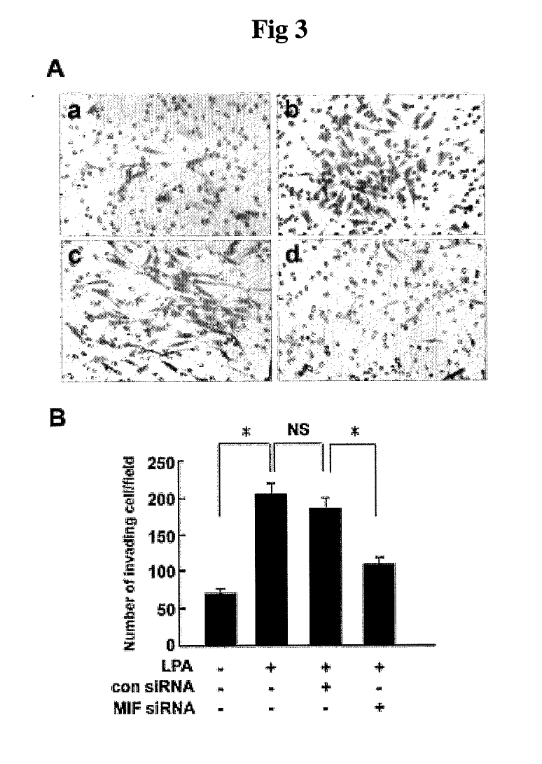

Invasion is the critical step in the metastatic cascade. To clarify the role of MIF siRNA in the tumor cell invasion in response to LPA, the inventors performed an in vitro invasion assay. Specifically, the effect of MIF siRNA (1 μg/ml) on the invasion of colon 26 cells was assessed using an in vitro invasion assay using a Matrigel-coated transwell assay as described in EXPERIMENTAL EXAMPLE 8. In FIG. 3A, cells migrated through the pore of membranes were stained with Diff Quick solution. The images shown are representative of three independent experiments. a, control without stimulation; b, stimulation with LPA (20 μM); c, stimulation with LPA (20 μM) after control siRNA treatment; d, stimulation with LPA after MIF siRNA treatment. In FIG. 3B, quantitative analysis of LPA-induced cell invasion and the effect of MIF siRNA (n=3). *Values are statistically significant (P<0.05). The cells were treated with MIF siRNA (1 μg/ml) or control siRNA for 24 h, and were cultured for another 24 h in the absence of serum prior to LPA stimulation. Following this, The inventors stimulated the cell with LPA (20 μM). The inventors found that LPA-induced migration and invasion was suppressed by MIF siRNA, whereas control siRNA did not affect the degree of tumor cell migration (FIG. 3). These findings indicate that MIF may play a key role in the migration and invasion of colon 26 cells.

### Example 4

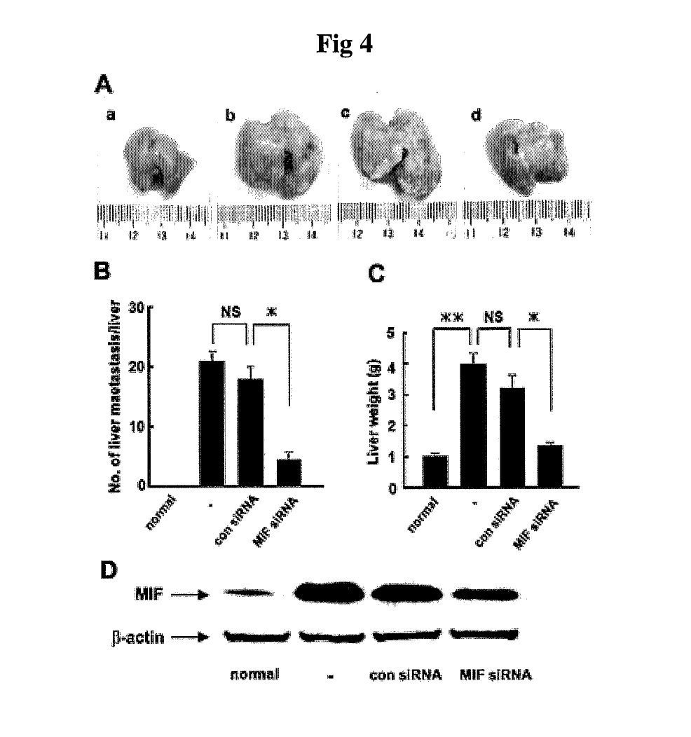

To examine usefulness of siRNA for the treatment of the colorectal metastasis, the inventors used the hepatic metastasis model. The metastatic abilities of colon 26 cells with MIF siRNA or control siRNA treatment (1 μg/ml) for 48 h were determined by the number of metastatic nodules present 14 days after inoculation through the portal vein. The results is shown in FIG. 4: A, Macroscopic images of metastatic nodules in the livers, (a) normal (no tumor cell injection); (b) positive control; (c) control siRNA; and (d) MIF siRNA; B, Quantitative analysis of the number of liver metastatic nodules, wherein *Values are statistically significant (P<0.05); C, Quantitative analysis of the liver weights, wherein *statistically significant (P<0.05) and **statistically significant (P<0.01); D, Western blot analysis using whole livers to assess MIF protein levels in livers with or without metastatic tumors. Macroscopically, the number and size of metastatic foci were decreased by MIF siRNA treatment as compared with cells treated with the control siRNA (FIG. 4A). The number of metastatic nodules in the livers of mice injected with MIF siRNA-treated cells was much smaller than that by treatment with control siRNA (FIG. 4B). The weight of the livers containing the metastatic nodules produced by the MIF siRNA-treated cells was less than that by treatment with the control siRNA (FIG. 4C). MIF siRNA dose-dependently affected the number of metastatic modules and the liver weight (data not shown). The inventors performed Western blot analysis using whole livers to assess MIF protein levels in livers with or without metastatic tumors. Each whole liver injected with colon 26 cells treated with or without siRNA was homogenized and subjected to Western blot analysis. The inventors found that MIF siRNA decreased the MIF protein level of the livers with metastatic tumors (FIG. 4D). These data indicate that MIF siRNA has the potential to suppress tumor growth and metastasis.

### Example 5

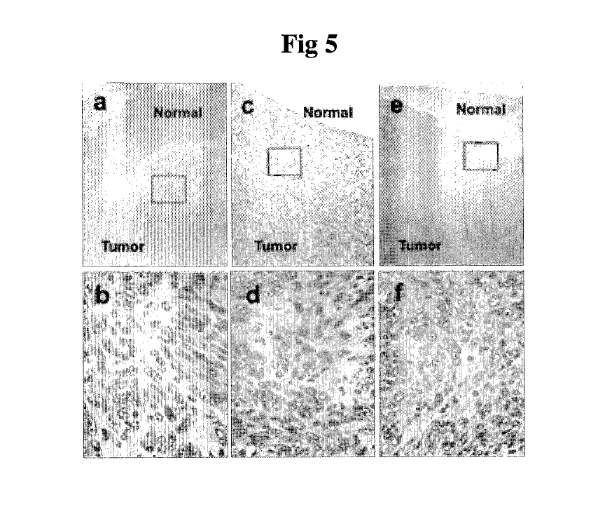

Histological changes in the liver surrounding metastatic foci were examined to evaluate the effectiveness of MIF siRNA. Specifically, the method was in accordance with EXPERIMENTAL EXAMPLE 6. In H-E staining, some mononuclear cells and vascularization were observed at the borders between normal tissue and tumor, when colon 26 cells without any pretreatment were injected through portal veins (positive controls) (FIGS. 5a, b). Similarly, significant vascularization was identified around the metastaic foci when colon 26 cells pretreated with the control siRNA were injected (conrols) (FIGS. 5c, d). On the hand, vascularities were markedly reduced between normal and tumor tissues treated with MIF siRNA (FIGS. 5e, f).

### Example 6

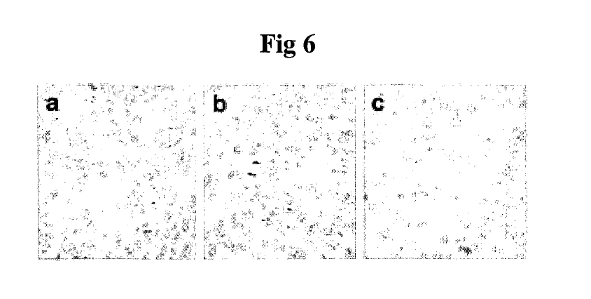

To confirm the neovasucularization, the inventors carried out immunohistochemical analysis using anti-CD31 antibody that reflects the vascular endothelial proliferation. Consistent with the results of FIG. 5, the inventors found the increased positive staining surrounding the metastatic foci of the cells non-treated (FIG. 6a) or treated with the control siRNA (FIG. 6b). In conrast, the number of CD31-positive-stained cells were markedly decreased (FIG. 6c).

### Example 7

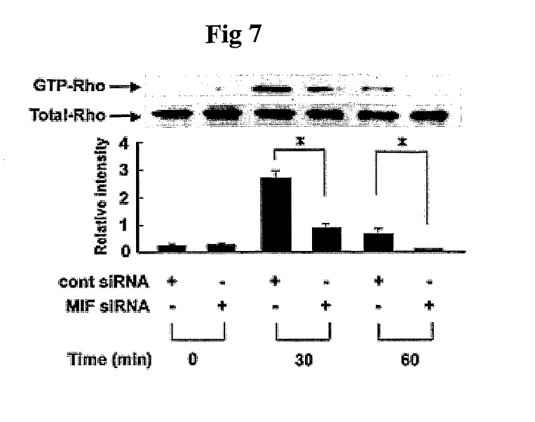

To confirm the inhibitory effect of MIF siRNA on LPA-induced migration and invasion, the inventors measured the intracellular levels of the GTP-bound, active form of Rho using the pull-down assay system. The level of the active form of Rho was elevated at 30 min, and gradually decreased after the addition of LPA at 60 min (FIG. 7). When the inventors treated the cells with MIF siRNA (1 μg/ml) for 48 h prior to LPA stimulation (20 μM, the elevation induced by LPA was markedly suppressed. These results indicate that MIF siRNA inhibited LPA-induced migration and invasion of colon 26 cells by suppressing the prenylation of the small GTP-binding protein Rho.

### Example 8

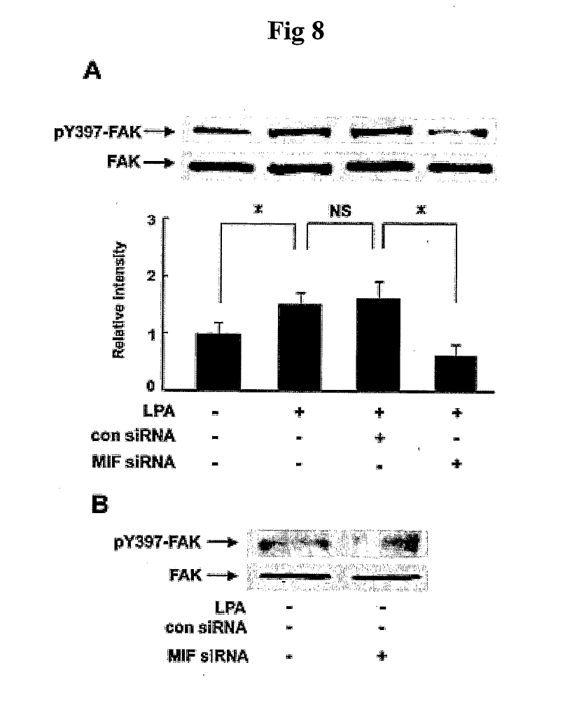

The inventors analyzed the effect of MIF siRNA on the phosphorylation of FAK by Western blot analysis. In preliminary experiments, the inventors assessed the time course of the phosphorylation of FAK induced by LPA. By the pretreatment of MIF siRNA (1 μg/ml) for 48 h, the inventors found that tyrosine-phosphorylation of FAK was significantly elevated at 30 min after LPA treatment (20 μM) (FIG. 8). Following this, the inventors found that the tyrosine phosphorylation of FAK in response to LPA was markedly suppressed. Similar results were obtained at 15 min and 60 min (data not shown). The total amounts of FAK proteins are shown at the bottom of each lane. The results demonstrate that MIF siRNA inhibited the Rho-mediated activation of actomyosin contractility and tyrosine phosphorylation of focal adhesion proteins by suppressing the prenylation of Rho.

### Example 9

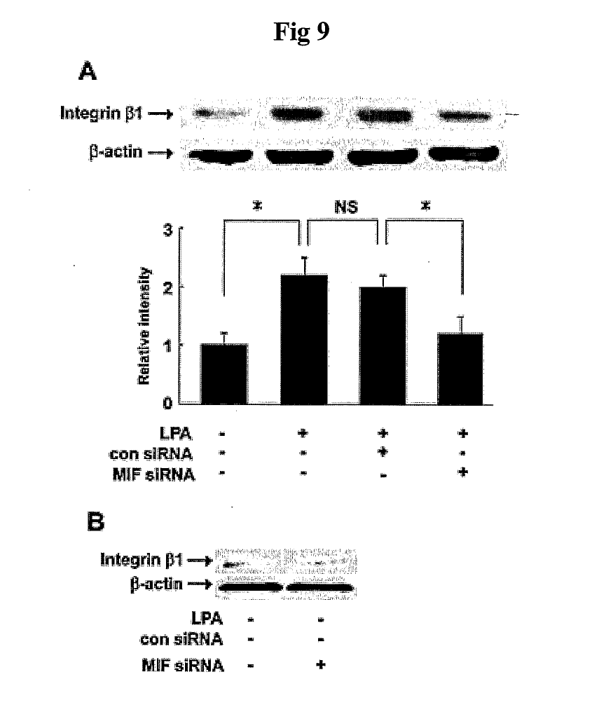

The inventors analyzyed the effect of MIFsiRNA on the LPA-induced integrin β1 expression by western blot analysis. After colon 26 cells were treated with MIF siRNA (1 μg/ml) or control siRNA for 48 h, the cells were cultured in the absence or presence of LPA for 24 h. Cells were lysed and the lysates (20 μg) were subjected to immunoblot analysis using anti-integrin β1, followed by peroxidase-conjugated secondary antibody and enhanced chemiluminescence detection (ECL detection). The inventors found that LPA induced the expression of integrin β1 (FIG. 9). When the cells were treated with MIF siRNA, the expression of integrin β1 was markedly suppressed. These results indicate that MIF plays an important role for the integrin signaling in the event of LPA-induced colon 26 cells adhesion and invasion.

### Example 10

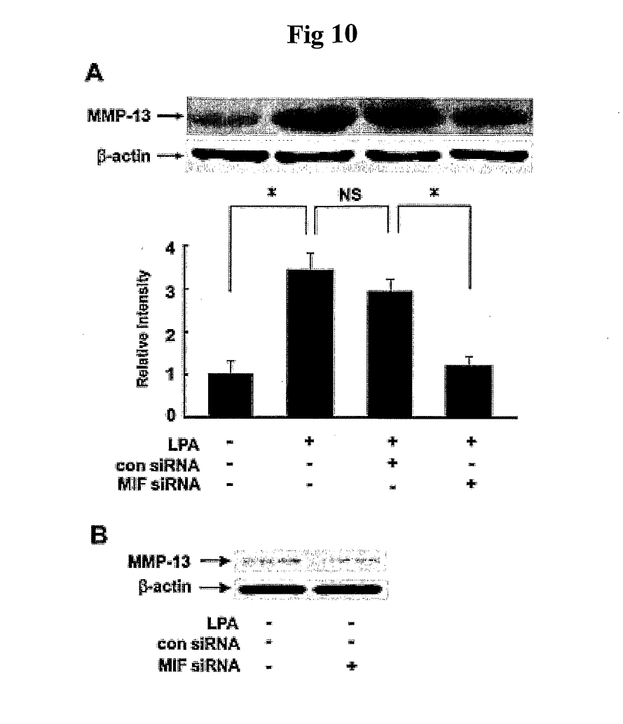

MMP is considered to be one of the important molecules involved in the induction of tumor invasion and metastasis. To examine the potential effect of MIF on MMP-13 expression in promoting tumor invasion and metastasis, the inventors evaluated the effectiveness of MIF siRNA on MMP-13 production in response to LPA. Specifically, after treatment with MIF siRNA (1 μg/ml) or control siRNA for 48 h, the cells were treated with LPA (20 μM) for 24 h, and the cell lysates were subjected to immunoblot analysis using anti-MMP-13 antibody, followed by ECL detection. The result shown is representative of at least three independent experiments. As shown in FIG. 10, the inventors found that MMP-13 production was remarkably suppressed by MIF siRNA treatment (1 μg/ml) for 48 h prior to LPA stimulation (20 μM).

### INDUSTRIAL APPLICABILITY

Effective treatment of tumor invasion and metastasis constitutes the biggest challenge in clinical oncology. The inventors here demonstrated that tumor cell motility induced by LPA, a unique bioactive lysophospholipid, was significantly inhibited by down-regulation of MIF using RNAi, a novel gene-silencing technique. Moreover, MIF siRNA markedly inhibited LPA-induced invasion of murine colon cancer cells by attenuating the activation of Rho, and thereby reducing the transmigration and focal adhesion assembly. Taken together, these results strongly indicate that MIF could be involved in the mechanism of tumor-cell growth as well as in LPA-induced tumor invasion and metastasis. This may provide the basis for a new therapy that controls the metastasis of colon cancer.

1. A pharmaceutical agent for preventing metastasis of cancer, which comprises a substance that inhibits expression of macrophage migration inhibitory factor (MIF).

2. A pharmaceutical agent according to claim 1, wherein the substance is selected from siRNA of MIF, antisense of MIF, ribozyme of MIF and a compound that inhibits expression of MIF.

3. A pharmaceutical agent according to claim 1, wherein the substance is siRNA of MIF.

4. A pharmaceutical agent according to claim 1, wherein the cancer is prostatic adenocarcinoma or colon cancer.

5. A method of preventing metastasis of cancer, which is characterized by using a substance that inhibits expression of MIF.

6. A method according to claim 5, wherein the substance is selected from siRNA of MIF, antisence of MIF, ribozyme of MIF and a compound that inhibits expression of MIF.

7. A method according to claim 5, wherein the substance is siRNA of MIF.

8. A method according to claim 5, wherein the cancer is prostatic adenocarcinoma or colon cancer.

9. Use of a substance that inhibits expression of MIF for manufacturing a pharmaceutical agent for preventing metastasis of cancer.

10. Use according to claim 9, wherein the substance is selected from siRNA of MIF, antisence of MIF, ribozyme of MIF and a compound that inhibits expression of MIF.

11. Use according to claim 9, wherein the substance is siRNA of MIF.

12. Use according to claim 9, wherein the cancer is prostatic adenocarcinoma or colon cancer.

13. A pharmaceutical agent according to claim 2, wherein the cancer is prostatic adenocarcinoma or colon cancer.

14. A pharmaceutical agent according to claim 3, wherein the cancer is prostatic adenocarcinoma or colon cancer.

15. A method according to claim 6, wherein the cancer is prostatic adenocarcinoma or colon cancer.

16. A method according to claim 7, wherein the cancer is prostatic adenocarcinoma or colon cancer.

17. Use according to any one of claim 10, wherein the cancer is prostatic adenocarcinoma or colon cancer.

18. Use according to any one of claim 11, wherein the cancer is prostatic adenocarcinoma or colon cancer.
